(19) United States
(12) Patent Application Publication
(10) Pub. No.: US 2005/0220913 A1
(43) Pub. Date: Oct. 6, 2005

(54) Novel in-expensive and efficient process for isolation of imperatorin, a potent inducible nitric oxide synthase inhibitor and anti-inflammatory drug candidate from Aegle marmelos Correa

(75) Inventors: Mangala Gowri Ponnapalli, Trivandrum (IN); Maheswari Muralidharan Jeeja, Trivandrum (IN); Madam Vadirajan Venugopalan Velupparambu, Trivandrum (IN); Arumugam Chami, Trivandrum (IN)

Correspondence Address: LADAS & PARRY, 26 WEST 61ST STREET, NEW YORK, NY, 10023, US

(73) Assignee: Council of Scientific and Industrial Research

(21) Appl. No.: 10/815,107

(22) Filed: Mar. 31, 2004

(51) Int. Cl. C07D 493/02; A61K 35/78

(52) U.S. Cl. 424/777; 549/282

(57) ABSTRACT

The present invention relates to a process for isolating imperatorin, an anti-first-pass effective low molecular weight linear furanocoumarin from the mature/immature mesocarp fresh/dry Aegle marmelos Correa fruits. Immunologically important phytosterols enriched fraction is a useful by-product of this process, which might enter this new century as therapeutics for targeting specific sites in the immune system.

The present invention relates to “A NOVEL IN-EXPENSIVE AND EFFICIENT PROCESS FOR ISOLATION OF IMPERATORIN, A POTENT INDUCIBLE NITRIC OXIDE SYNTHASE INHIBITOR AND ANTI-INFLAMMATORY DRUG CANDIDATE FROM AEGLE MARMELOS CORREA”. This invention leads to a process for the isolation of imperatorin, an anti-first-pass effective low molecular weight linear furanocoumarin from the mature/immature mesocarp of fresh/dry Aegle marmelos Correa fruits. Immunologically important phytosterols enriched fraction is a useful by-product of this process, which might enter this new century as therapeutics for targeting specific sites in the immune system.

Aegle marmelos Correa is a moderately sized, slender, aromatic tree, 6.0-7.5 m in height and 90-120 cm in girth, native to the Indian sub continent. It is growing wild throughout the deciduous forests of India and very much found in the sub-Himalayan forests, Central and South India. It is now naturalized in Sri Lanka, Pakistan, Bangladesh, Myanmar, Thailand and most of the southeastern Asian countries. It is known as Bael and Bel in Hindi, Assamese, Bengali; Marwari, Bili in Gujarati, Koovalam and Vilvam in Tamil, Bilvamu, Maredu in Telugu and Bel in Urdu and Stone Apple in English. Aegle belongs to one of the monotypic genera of orange sub family Aurantioideae, tribe Clauseneae and sub tribe Balsamocitrinae and family (Rutaceae).

Fruits of Aegle marmelos Correa are 5.0-7.5 cm in diameter, globose, oblong pyriform, rind grey or yellow; pulp sweet, thick yellow, orange to brown in color. Seeds are numerous and arranged in the cells surrounded by a slimy transparent mucilage. The unripe or half-ripe fruit is regarded as astringent, digestive and stomachic. The fruit is reputed to be a valuable Ayurvedic medicine for chronic diarrhea and dysentery and said to act as a tonic for heart and brain. Clinical trails of unripe fruits showed anti-viral activity against Ranikhet disease virus, hypoglycaemic activity and significant results against parasites. The pulp, diluted with water and added with requisite amount of sugar and tamarind, forms a delicious cooling drink. The Bael fruit is one of the most nutritious edible fruits, rich in carotenoids, riboflavin and pectin, used for preparation of a number of products like candy, squash, toffee, slab, pulp powder and nectar (The Wealth of India vol. 5, 1990-94 and Compendium of Indian Medicinal Plants vol. 1-5, 1962-1998).

### FIELD OF INVENTION

The present invention “A NOVEL IN-EXPENSIVE AND EFFICIENT PROCESS FOR ISOLATION OF IMPERATORIN, A POTENT INDUCIBLE NITRIC OXIDE SYNTHASE INHIBITOR AND ANTI-INFLAMMATORY DRUG CANDIDATE FROM AEGLE MARMELOS CORREA” relates to a process for the isolation of a compound ‘imperatorin’, useful as a potential inducible nitric oxide synthase (iNOS) inhibitor and anti-inflammatory drug candidate from fruit pulp of mature/immature Aegle marmelos Correa (Rutaceae). Imperatorin belongs to the linear furanocoumarin group of compounds. Imperatorin (9-[3-methyl-2-butenyl)-oxy]-7H-furo[3,2-g] [1] benzopyran-7-one) is a furano derivative of benzo-alpha-pyranocoumarin and is found in the plants in the free state.

### BACKGROUND OF INVENTION

Furanocoumarins (also called furocoumarins and sometimes designated psoralens, after one of the best known furanocoumarins), a class of widely occurring phenolic compounds, especially abundant in citrus fruits, these compounds are ubiquitous in higher plants. These are found in approximately 15 plant families, of which members of the family Apiaceae (Umbelliferae) are well known producers of furanocoumarins followed by Rutaceae, Moraceae and Leguminasae. Several of these plants eg. lemon, cilantro, celery, pastinak, parsley and carrots are part of the human diet. Furanocoumarins have several interesting biological activities, such as analgesic, antiinflammatory, antibacterial, antiviral, anticoagulant in addition to their well-known photosensitizing effect. Imperatorin isolated from roots of Angelica dahurica (Umbelliferae) was found to induce apoptosis in human promyelocytic leukaemia, HL-60 cells. DNA fragmentation assay, morphology-based evaluation and flow cytometric analysis demonstrated that imperatorin at micromolar concentrations was able to trigger apoptosis of HL-60 cells (Hyun-Ock et al., Imperatorin, a furanocoumarin from Angelica dahurica (Umbelliferae), induces cytochrome c-dependent apoptosis in human promyelocytic leukaemia, HL-60 cells; Pharmacology & Toxicology, vol. 91, no. 1, pp 40-48, 2002). Imperatorin showed strong activity of HL-60 cells by nitro blue tetrazolium (NBT) method (Kawa et al., Effect of coumarins on HL-60 cell differentiation; Anticancer research, vol. 20, No. 4, pp 2505-2512, 2000). Imperatorin isolated from chinese herb I. Saposhnikovia divaricata (Turcz) Schischk is potential inducible nitric oxide synthase (iNOS) inhibitor, displaying IC₅₀ value of 17.3 μg/ml for inhibition of nitrite production, which can be used as cancer chemopreventing agent (Yang et al. Inducible nitric oxide synthase inhibitor of the Chinese herb I. Saposhnikovia divaricata (Turcz) Schischk; Cancer Letters, vol. 145, pp 2505-2512, 1999).

Imperatorin is the main component of Yuanhu analgesic capsule (0.66 to 0.72 mg/capsule), which is made of Yuanhu and Radix Angelicae dahuricae, which is recorded in the eighth volume (1993) of medicine standards published by the Ministry of Health of the People's Republic of China (Wenxiang et al. Determination of imperatorin in Yuanhu analgesic capsule by RP-HPLC;. Chemical Journal on Internet, vol. 3, No. 11, pp 56-59, 2001). It is therefore, valuable to develop a potent and economically fasible natural source of inhibitor of iNO for potential therapeutic and commercial use in the future.

Imperatorin is widely distributed in the plant kingdom and can be isolated from the different parts (especially fruits, seeds and roots) of the plant. It is isolated from roots of Imperatoria osthruthium L., seeds of Angelica archangelia L., and fruits of Pastinaca sativa L. (The Merck index, XII edition, p4960). Imperatorin is also isolated from fruits of Anethum graveolens, Angelica archangelica, Anthriscus cerefolium, Apium graveolens, Carum carvi, Coriandrum sativum, Cuminum cyminum, Daucus carota, Foeniculum vulgare, Heracleum sphondylium, Levisticum officinale, Pastinaca sativa, Petroselinum crispum and Pimpinella anisum (Ceska et al. Photoactive furocoumarin in fruits of some umbellifers; Phytochemistry. vol. 26, No. 1, 165-169, 1986). The presence of Imperatorin in Aegle marmelos Correa has been reported only from India (Asima Chatterdee et al., Isolation of allo-imperatorin and β-sitosterol from the fruits of Aegle marmelos Correa; J.Ind. Chem. Soc., vol. 34, No. 3, pp 228-230, 1957), though the plant is grown in southeastern Asian countries. Nearly 40 other plant species of different families are also reported to contain imperatorin.

Despite the plant Aegle marmelos Correa having widely used in India for medicinal purposes as well as for making cooling drink, not many patents exist related to Aegle marmelos. A few patents, which exist, relate to herbal catalytic compositions using Aegle marmelos for pollution control and energy saving of fuel used for automobile, and antidiabetic compositions (IPR Bulletin, Vol. 7, No. 3-4, March-April 2001).

In India, Asima Chatterjee (Asima Chatterjee et al., Isolation of allo-imperatorin and β-sitosterol from the fruits of Aegle marmelos Correa; J. Ind. Chem. Soc., vol. 34, No. 3, pp 228-230, 1957) reported isolation of imperatorin from Aegle marmelos in 1957, wherein crystallization of benzene solution of a concentrated alcoholic extract of the fruit pulp, yielded allo-imperatorin in 0.003% and mother liquor was chromatographed over a column of Brockmann alumina (12 cm×1 cm) using gradient mixures of pet, ether and benzene (1:4) furnished imperatorin in 0.006% and β-sitosterol mixture, in 0.00125% yield. The major drawback of the method is the use of benzene as a solvent for partition of methanolic extract for the isolation of imperatorin, which otherwise is highly carcinogenic and banned. It is not only timeconsuming process but also an expensive one besides its extremely poor yield.

The content of imperatorin isolated from Magnolia pterocarpa Roxb is 0.003% (Talapatra et.al., Polyphenolic constituents of Magnolia pterocarpa Roxb. J. Ind. Chem. Soc., vol. 60, 1983).

It has been observed that several other compounds frequently are found together with furanocoumarins, making their isolation difficult. In general, isolation of furanocoumarins depends initially upon successive extraction of dried plant with commonly used solvents of increasing polarities (pet, ether, benzene, ether, methanol and ethanol etc.) It has been observed that non-polar solvents for extraction of the plant materials were employed, resulting in less recovery of furanocoumarins. Still, polar solvents (methanol and ethanol) used for the extraction of furanocoumarins resulted in a, higher amount of total extract having more colour and fatty material. The separation of fatty and colouring matter is a difficult task. In the present invention, we have selected halogenated solvent for extraction, which yielded a higher amount of imperatorin with less amount of colouring and fatty material. Also, recovery of the pure solvent for reuse is much easier.

No cost-effective process exists today for the isolation of Imperatorin directly from fresh/dry mesocarp of Aegle marmelos Correa. There are no reports on earlier attempts for direct processing of fresh/dry fruits of Aegle marmelos Correa with vacuum liquid chromatography for isolation of imperatorin. As such no plant with high content of imperatorin for commercial exploitation or any large-scale process for the isolation of pure imperatorin has been reported.

### OBJECTS OF THE PRESENT INVENTION

The main objective of the present invention is to provide a novel cost-effective, inexpensive, efficient and commercially feasible process for the isolation of imperatorin from fresh/dry mesocarp of mature/immature Aegle marmelos Correa,

Another objective of the present invention is to provide a method for obtaining immunologically important phytosterols enriched fraction as by-product.

Another objective of the present invention is to explore Aegle marmelos Correa as a commercial source with high content of imperatorin,

Another objective of the present invention is to select a cost-effective solvent for selective extraction of imperatorin.

Still another objective of the imperatorin is to develop an economically feasible process for the isolation of pure imperatorin for therapeutic use.

### NOVELTY OF INVENTION

1. For the first time a commercially feasible process for isolation of imperatorin from Aegle marmelos Correa is reported in the present invention.

2. Identification of the fruit for isolation of imperatorin improved the yield and reduced the processing cost.

3. The extraction of fresh/dry fruit pulp directly with ethylenedichloride is more economical with a better yield of imperatorin of high purity as compared to the use of other solvents like methanol or ethanol and partitioning to halogenated solvents.

4. The selective transfer of imperatorin from alcoholic extract to the halogenated non-polar phase resulted in easy purification and isolation of pure imperatorin.

5. Crystallization of the imperatorin directly from the crude extract resulted in 60-70% yield of imperatorin before vacuum liquid chromatography.

6. The process allows the reuse of solvents and silica gel.

7. The process allows the phytosterols enriched fraction as a useful by-product.

These advantages are of significant economic value and easy to perform on a large commercial scale production of imperatorin.

### DETAILED DESCRIPTION OF THE INVENTION

The present invention provides a novel process for the isolation of compound imperatorin, which is used as potential inducible nitric oxide synthase inhibitor and ant-inflammatory drug candidate from mature/immature fruits of Aegle marmelos Correa, the said process comprising:
a) extraction of fresh/dried powdered pulp with halogenated solvent directly or with monohydric alcohol at ambient temperature for 24 to 48 hrs. (pulp:solvent 1:3 to 1:6) or with halogenated solvent or monohydric alcohol in a Soxhlett apparatus for 6 to 12 hrs. (pulp:solvent 1:4),
b) concentration of the extracted alcoholic solvent up to 10 to 30% of its original volume under vacuum,
c) partitioning the concentrated alcoholic extract with halogenated solvent to transfer imperatorin in non-polar solvent,
d) drying the extracted miscella obtained directly or by partition, over anhydrous sodium sulphate and evaporating the solvent,
e) crystallizing the concentrate in pet-ether-dichloromethane mixture and filtering the crystals,
f) concentrating the filtrate and subject to vacuum liquid chromatography on silica gel,
g) eluting imperatorin in pet-ether-ethyl acetate mixture to afford phytosterols enriched fraction and pure imperatorin,
h) crystallizing the fractions containing pure imperatorin compound.

In one embodiment of the present invention, mature/immature fresh fruit pulp or dried powdered fruit pulp of Aegle marmelos Correa were selected for fresh process or dry process for the isolation of imperatorin.

In another embodiment of the present invention, wherein screening on all the mature and immature fruits of Aegle marmelos Correa for the isolation of imperatorin was carried out by RP-HPLC in fresh and dry processes using different solvents.

Yet other embodiment of the present invention, wherein the yield of imperatorin from Aegle marmelos Correa is very high as compared to other reported plants.

In an embodiment of the present invention the halogenated solvent used for direct extraction or partition is selected from dichloromethane, chloroform, carbon tetrachloride and ethylenedichloride.

In another embodiment of the present invention, the monohydric alcohol used for extraction is either methanol or ethanol.

Still another embodiment of the present invention, wherein halogenated solvent was used for the isolation of imperatorin, which resulted in yielding imperatorin with less amount of colour and fatty material.

Yet other embodiment of the present invention, wherein the furanocoumarins are selectively extracted with non-polar chlorinated solvent directly or transfer of furanocoumarin from the alcoholic phase with chlorinated solvent (carbon tetrachloride, methylene dichoride and ethylenedichloride),

Yet other embodiment of the present invention, wherein most of the colour and fatty material in the imperatorin is left out in the polar phase, thereby enriches the crystallization (60-70%) of imperatorin in non-polar phase.

Still another embodiment of the present invention, wherein the imperatorin left out in the mother liquor after crystallization is subjected to vacuum liquid chromatography over silica gel (230-400 mesh) in the ratio of 1:4 to 1:6 for complete isolation of pure imperatorin.

Yet other embodiment of the present invention, wherein the partition of imperatorin from alcoholic extract to non-polar solvent reduces the bulkiness of the crude extract by 65-75%, which in turn requires fewer amoLunts of silica gel and solvent in the process.

Yet another embodiment of the present invention, imperatorin is crystallized from the solvent, which is selected from pet-ether, acetone and dichloromethane and mixtures thereof.

Still another embodiment of the present invention, yield of imperatorin, isolated from fresh mature fruits is in the range of 0.74% to 1.43% (dry weight basis) by direct process of 2 days cold percolation with EDC/DCM (pulp:solvent 1:3).

One another embodiment of the: present invention, yield of imperatorin isolated from dry mature fruits is in the range of 1.24 to 1.66% (dry weight basis) by direct process of 2 days percolation with EDC/DCM (pulp:solvent 1:3).

One another embodiment of the present invention, yield of imperatorin isolated from the fresh mature fruits is in the range of 2.19% and 2.15% (dry weight basis) by cold percolation for 2 days with EDCIDCM (pulp:solvent 1:6).

One another embodiment of the present invention, yield of imperatorin isolated from fresh mature fruits is 1.92%/2.29% (dry weight basis) by process of DCM/EDC partition of rnethanolic extract.

One another embodiment of the present invention, yield of imperatorin isolated from immature fruits is in the range of 0.52% by dry process of DCM parition of methanolic extract.

One another embodiment of the present invention, yield of imperatorin isolated from mature fruits (3.12%) immature fruits (0.89%) and ripe fruits (1.71%) by extraction in a Soxhllett apparatus for 6-12 hrs. with ethylenedichloride.

The present invention is to provide a novel and cost-effective process for the isolation of imperatorin from Aegle marmelos Correa to overcome the drawbacks of hitherto known process from other sources. The invention more particularly provides a process, which gives a cheaper and higher yield of potent inducible nitric oxide synthase inhibitor and anti-inflammatory drug candidate, imperatorin from the natural source.

### EXAMPLE 1

Selection of Fruits for Extraction

A comparative study of imperatorin content in the immature, mature and half-ripe stages of fresh fruit pulp of Aegle marmelos Correa was done. 50 g of dried sample of moisture content (2.5 to 4%) was extracted with ethylenedichloride in a Soxhlett apparatus for 6 to 12 hrs. The extracted miscella was filtered and dried over anhydrous sodium sulphate and the solvent was evaporated to dryness under vacuum, The extract (1 mg) was dissolved in 5 ml HPLC grade methanol. The content of imperatorin in each sample was estimated by LC8A Shirnadzu HPLC equipped with UV detector under the following operating conditions: mobile phase methanol:water (50:50); flow rate 1 ml/min, detection at 300 nm, column-C18 R-ODS-S-A 5 μm. Quantification was performed using a calibration curve of the standard imperatorin estimated in different ripening stages of fruits are as follows; immature (0.89%), mature (3.12%) and ripe (1.71%) of purity in the range of 54% to 62%.

### EXAMPLE 2

Selection of Solvent for Extraction

Fresh crushed sample (100 gm) of Aegle marmelos Correa with moisture content 71.5% was extracted with 300 ml of different solvents (carbon tetrachloride, dichloromethane, ethylenedichloride and ethylenedichloride:methanol (9:1) for cold percolation for 24 hrs at ambient temperature. Each extract was filtered and dried over anhydrous sodium sulphate and concentrated under vacuum. Each extract (1 mg) was dissolved in 5 ml HPLC grade methanol and estimated the imperatorin content by HPLC method as described in example 1. Screening of all the mature, immature and ripe fruits of Aegle marmelos Correa for fresh and dry processes with different solvents for imperatorin content was carried out by RP-HPLC. The content of imperatorin obtained in different processes are as follows: fresh process: direct EDC (0.75%), DCM (0.82%), EDC:MeOH (9:1) (0.66%) and CCl₄ (0.38%) on dry weight basis after 24 hrs. cold percolation (pulp:solvent 1:3) at room temperature of mature fruits of moisture 71.5%. Further prolonged percolation for a day increased the content of imperatorln viz. EDC by (1.43%), DCM by (1.24%) and EDC:MeOH (9:1) by (1.22) on dry weight basis. EDC and DCM percolation (pulp:solvent 1:6) of fresh mature fuit pulp of moisture 68% afforded imperatorin content 2.19% and 2.15% after 2 days continuous percolation.

Dry process: The content of imperatorin in the dry process of mature fruits is as follows: EDC (1.31%), DCM (1.24%) after one day percolation, further improved to (1.66%), (1.56%) after 2 days percolation on dry weight basis of the purity in the range of 65% to 70% EDC/DCM partition of methanolic extract of fresh mature fruit pulp contains (1.92%)/(2.29%) on dry weight basis of the purity in the range of 40% to 50%.

### EXAMPLE 3

Extraction and Isolation of Imperatorin from Mature Fruit Pulp

Mature fruits of Aegle marmelos Correa were depulped mechanically and smashed pulp of moisture content (68%) was percolated directly with dichloromethane [170.9 g fresh pulp contained 54.68% dry matter] or ethylenedichloride [399.4 g fresh pulp contained 127.8% dry matter] (pulp:solvent 1:6) for 24 to 48 hrs at ambient temperature. The total extracted miscella was dried over anhydrous sodium sulphate and concentrated under vacuum. The concentrate [DCM 2.85 g/EDC 3.93 g] was dissolved in n-hexane-dichloromethane mixture (15 to 20 ml) and kept for crystallization 4-5 hrs. in a refrigerator (0-4° C.) The crystalline compound was filtered and dried to get pure imperatorin. The process of crystallization was repeated three times to get (60 to 70%) pure imperatorin compound (1 g, by DCM/2.0254 g by EDC). The remaining imperatorin in the filtrate was isolated by vacuum liquid chromatography over silica gel in the ratio of (1:5) and imperatorin with 20% to 40% EtOAc in n-hexane. The imperatorin containing fractions were pooled and evaporated and subjected to crystallization, which afforded pure imperatorin (0.176 g by DCM/0.7729 g by EDC). Thereby (85%) of pure imperatorin was isolated from 170.9 gm of fresh mature fruit pulp of 2.15% imperatorin while (90%) pure imperatorin was isolated from 399.4 g of fresh mature crushed pulp of imperatorin content 2.19% using dichloromethane/ethylenedichloride cold percolation. Immunologically important phytosterols mixture enriched fraction (0.04 to 0.16%) was obtained as a useful by-product of this process.

The imperatorin was identified by m.p., IR, UV, ¹H, ¹³C NMR and mass spectral data as reported in the literature.

### EXAMPLE 4

Extraction and Isolation of Imperatorin from Immature Fruit Pulp of Aegle marmelos Correa.

Immature fruits of Aegle marmeols Correa were depulped mechanically, cut into thin slices and dried under shade. Dry powdered pulp (111 g) of moisture content (3.5%) was extracted with methanol for 6 to 12 hrs. in a Soxhlett apparatus. The total extract was concentrated to 10 to 30% of its original volume under vacuum. The concentrated methanolic extract was partitioned with dichloromethane (5 times, 100 ml). The dichloromethane extract (3.66 g) was dried over anhydrous sodium sulphate and evaporated under vacuum. Imperatorin was isolated from this residue as described in example 3. Thereby (82%) pure imperatorin (0.5668 g) was isolated from 111 g of shade dried fruit pulp of 0.52% imperatorin content by DCM partition dry process of methanolic extract. Phytosterol mixture (0.10%) was obtained as a useful by-product.

### ADVANTAGES OF THE INVENTION

1. This invention is a cost effective and high yielding process for isolating imperatorin from the mature/immature fruits of Aegle marmelos Correa.

2. The extraction of fresh/dry fruit pulp directly with ethylene dichloride is more economic with a better yield of imperatorin of high purity as compared to the use of other solvents like methanol or ethanol and partitioning to halogenated solvents

3. The selective transfer of imperatorin from alcoholic extract to the halogenated non-polar phase resulted in easy purification and isolation of pure imperatorin.

4. The purification of the crude extract resulting in crystallization of 60-75% of imperatorin before vacuum liquid chromatography.

5. The process allows the reuse of solvents and silica gel.

6. The process allows the phytosterols enriched fraction as a useful by-product.

7. These advantages are of significant economical value and easy to perform on a large commercial scale production of imperatorin.

The present invention is to provide a novel and cost-effective process for the extraction and isolation of imperatorin from Aegle marmelos Correa to overcome the drawbacks of hitherto known, process from other sources. The invention more particularly provides a process, which gives a cheaper and higher yield of potent inducible nitric oxide synthase inhibitor and anti-inflammatory drug candidate, imperatorin from the natural source.

1. The present invention relates to a process for isolating imperatorin, an anti-first-pass effective low molecular weight linear furanocoumarin from Aegle marmelos Correa fruits comprising the steps of:
a) extracting fresh/dried powdered material of mature/immature pulp of Aegle marmelos Correa fruits directly with halogenated solvent at ambient temperature for 24 to 48 hrs. or with halogenated solvent or monohydyic, alcohol in a Soxhlett apparatus for 6 to 12 hrs, wherein a ratio of the pulp to the solvent is 1:6;
b) concentrating the extracted alcoholic solvent up to 10-30% by volume of its original extract under vacuum;
c) partitioning the concentrated alcoholic extract with halogenated solvent to transfer imperatorin in the non-polar halogenated solvent;
d) drying the the extracted portion obtained directly or by partition over anhydrous sodium sulphate and evaporating the solvent.
e) crystallizing the concentrates obtained from step (d) with a solvent and filtering the crystals to obtain a filtrate;
f) concentrating the filtrate;
g) subjecting the concentrated filtrate of step (f) to silica gel vacuum liquid chromatography;
h) eluting imperatorin from the concentrated filtrate of step (g) in a pet-ether-ethyl acetate mixture;
i) identifying the eluted fractions enriched with phytosterols mixture;
j) identifying the fraction containing the eluted imperatorin;
k) crystallizing the fractions containing imperatorin to obtain pure imperatorin

2. A process as claimed in claim 1, wherein the plant parts of Aegle marmelos used for the extraction of imperatorin are selected from the mature/immature/ripe fruit pulp;

3. A process as claimed in claim 1, wherein the halogenated solvent is selected from the group consisting of dichlorormethane, carbon tetrachloride and ethylenedichloride;

4. A process as claimed in claim 1, wherein the monohydric alcohol solvent is selected preferably either methanol or ethanol;

5. A process as claimed in claim 1, wherein the imperatorin is crystallized from the solvent, wherein the solvent is selected from the group consisting of pet-ether, dichloromethane, acetone and methanol mixtures thereof;

6. A process as claimed in claim 1, wherein the said isolation yields 0.75% to 3.12% imperatorin from mature fruits and 0.52% to 0.89% from immature fruits on dry weight basis;

7. A process as claimed in claim 1, wherein a ratio of the concentrated extract of step (g) to silica gel (230-400 mesh) is in the range of 1:4 to 1:6;

8. A process as claimed in claim 1, wherein the concentrated extract of step (g) subjected to vacuum liquid chromatography over silica eluting with the mixtures of pet-ether-ethyl acetate is in the range of 9:1 to 8.5:1.5 to get the fraction enriched with phytosterols mixture (0.04% to 0.17%);
